(19) United States
(12) Patent Application Publication
(10) Pub. No.: US 2003/0009109 A1
(43) Pub. Date: Jan. 9, 2003

(54) Bioelectrical impedance measuring apparatus

(76) Inventors: Takashi Yasujima, Tokyo (JP); Kentaro Kimura, Niiza-Shi (JP); Soji Kurata, Tsurugashima-Shi (JP); Masayuki Sato, Tokyo (JP)

Correspondence Address: MCDERMOTT, WILL & EMERY, 600 13th Street, N.W., Washington, DC, 20005-3096, US

(21) Appl. No.: 09/897,730

(22) Filed: Jul. 3, 2001

(51) Int. Cl. A61B 5/05; A61B 5/103; A61B 5/117

(52) U.S. Cl. 600/547; 600/587

(57) ABSTRACT

A bioelectrical impedance measuring apparatus in accordance with the present invention consists mainly of a housing, rod-like electrode members, a display device, and a weighing device. The electrode members each have a plurality of electrodes, and are included in the upper part of the housing. The display device has the capability of an operator panel and lies between the electrode members. The weighing device is included in the lower part of the housing. The housing accommodates a current supplying device, a voltage measuring device, and an arithmetic unit. The current supplying device supplies current to the electrodes. The voltage measuring device measures voltage at the electrodes. The arithmetic unit calculates a bioelectrical impedance value from the supplied current value and the measured voltage values. In practice, the bioelectrical impedance measuring apparatus includes a weighing device and measures bioelectrical impedance relative to current flowing between user's hands. A user need not have his/her feet bared. Nevertheless, the bioelectrical impedance can be measured highly precisely. Moreover, since electrode members are shaped like rods and arranged lengthwise, an error derived from a change in a posture caused by a difference in a height can be minimized.

### BACKGROUND OF THE INVENTION

1. Field of the Invention

The present invention relates to a bioelectrical impedance measuring apparatus that measures bioelectrical impedance.

2. Description of the Related Art

Conventional impedance measuring apparatuses fall into a foot type that includes a weighing device and a hand type that requires a user to manually enter his/her body weight.

In the conventional foot-type measuring apparatus, electrodes are exposed on a step base of the weighing device. For measurement, a user has to have his/her feet bared. This is rather discouraging to potential users. In contrast, the conventional hand-type measuring apparatus requires a user to manually enter his/her body weight. The precision in an entered body weight is controversial.

### SUMMARY OF THE INVENTION

The present invention attempts to solve the problems underlying in related arts. An object of the present invention is to provide a bioelectrical impedance measuring apparatus that enables accurate measurement without requiring a user to have his/her feet bared while making the most of the merits of both the foot type and hand type.

According to the present invention, there is provided a bioelectrical impedance measuring apparatus consisting mainly of a housing, rod-like electrode members, a display device, and a weighing device. The electrode members each have a plurality of electrodes, and are included in the upper part of the housing. The display device has the capability of an operator panel and lies between the electrode members. The weighing device is included in the lower part of the housing. The housing accommodates a current supplying device, a voltage measuring device, and an arithmetic means. The current supplying device supplies current to the electrodes. The voltage measuring device measures voltage at the electrodes. The arithmetic means calculates a bioelectrical impedance value from the supplied current value and the measured voltage values.

According to one aspect of the present invention, the electrode members are located at both side edges of the housing.

According to another aspect of the present invention, the electrode members are shaped circularly.

According to still another aspect of the present invention, the electrode members are shaped like horizontal rods.

### BRIEF DESCRIPTION OF THE DRAWINGS

FIG. 1 is a perspective view of a measuring apparatus;

FIG. 2 shows the structure of a rod-like electrode member;

FIG. 3 is an electrical block diagram of the measuring apparatus;

FIG. 4 is a flowchart describing a control sequence the measuring apparatus follows;

FIG. 5A to FIG. 5F show screen images displayed on the display screen of a display device;

FIG. 6A and FIG. 6B show screen images displayed on the display screen of the display device;

FIG. 7 shows a screen image displayed on the display screen of the display device;

FIG. 8 shows another embodiment having electrodes that have a different shape; and

FIG. 9 shows still another embodiment having electrodes that have a different shape.

### DESCRIPTION OF THE PREFERRED EMBODIMENTS

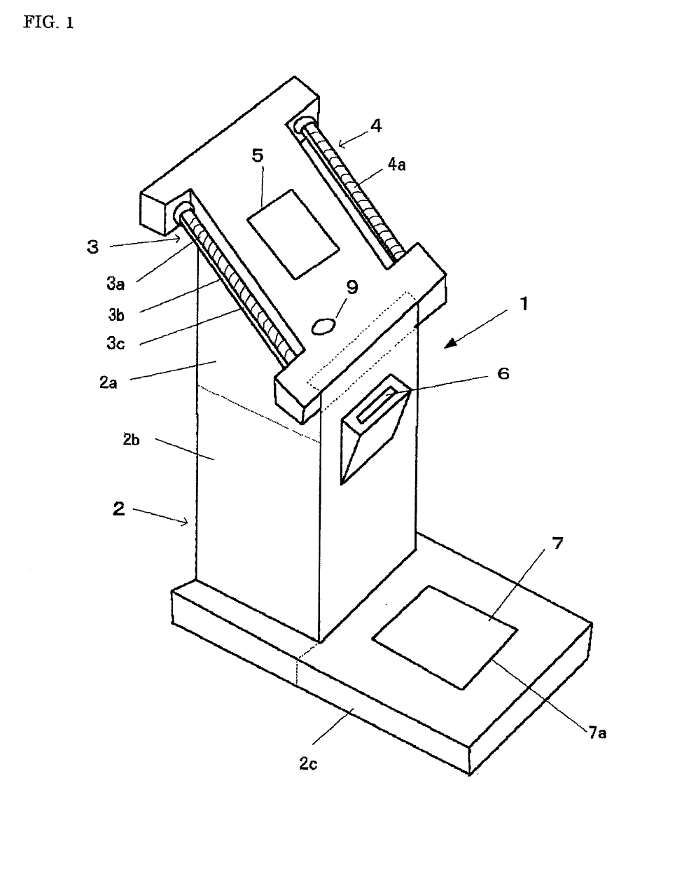

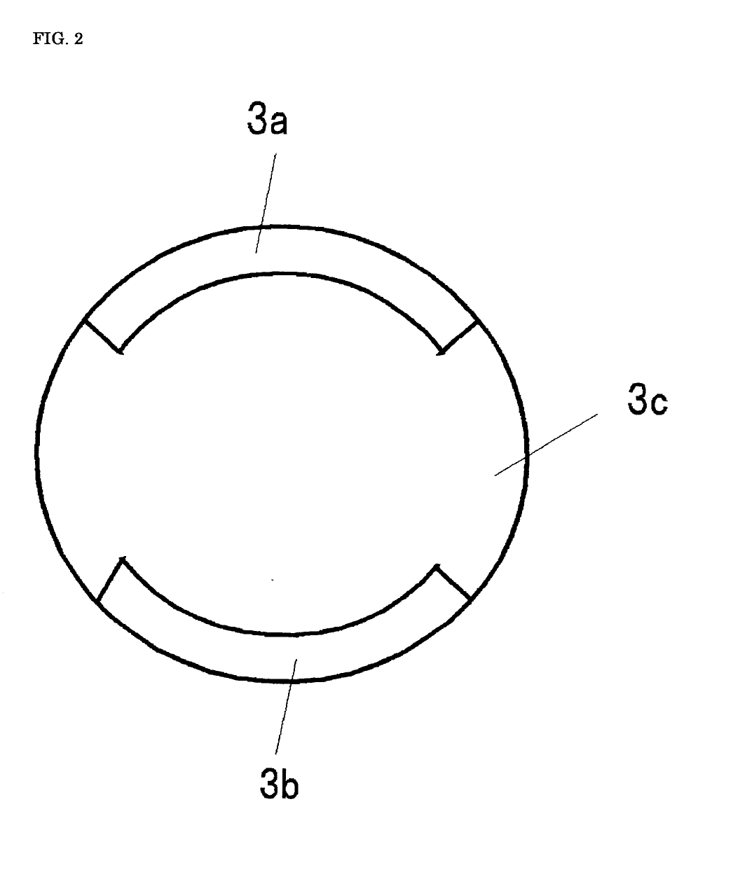

Embodiments will be described in conjunction with the drawings. FIG. 1 is a perspective view of a measuring apparatus. The measuring apparatus 1 is shaped substantially like letter L and has a weighing device 7 incorporated in a lower part 2c thereof. The measuring apparatus is supposed to be installed by the side of a shelf in a convenience store or drugstore or by the side of an entrance of the convenience store or drugstore. The weighing device 7 is a known one. Rod-like electrode members 3 and 4 are located at both side edges of an upper part 2a of the measuring apparatus 1. The electrode member 3 or 4 is composed of an electrode 3a or 4a through which current flows into a user's body, and an electrode 3b or 4b at which voltage is measured. FIG. 2 is a cross-sectional view of the electrode member 3. An insulator 3c is interposed between the electrodes 3a and 3b. The electrode member 4 has the same cross section as the electrode member 3 does. Furthermore, a display input device 5 including a touch panel type liquid crystal display device is interposed between the electrode members 3 and 4 and included in the upper part 2a of the measuring apparatus 1. An intermediate part 2b linking the upper part 2a of the apparatus 1 and the lower part 2c thereof includes a printer 6 that prints a result of measurement.

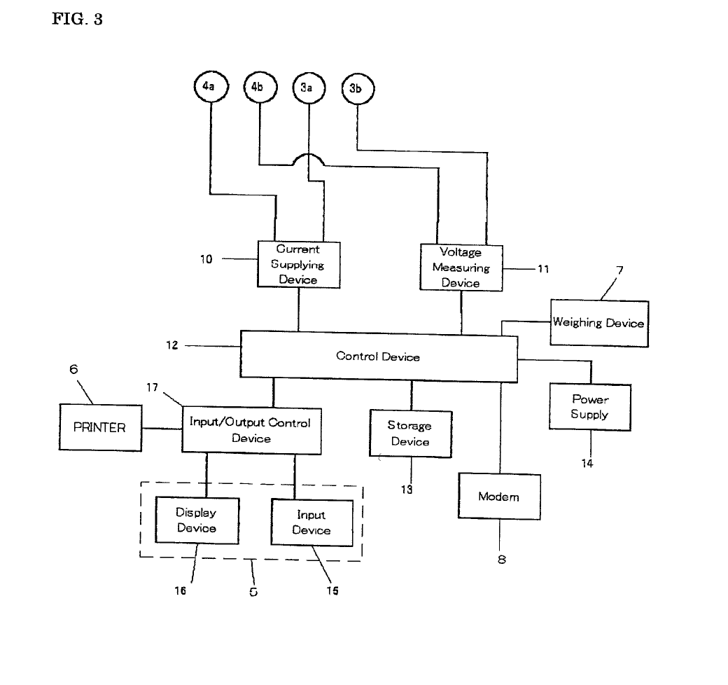

FIG. 3 is an electrical block diagram of the measuring apparatus 1. The electrodes 3a and 4a to be touched with the right and left hands are connected to a current supplying device 10. The electrodes 3b and 4b are connected to a voltage measuring device 11. The current supplying device 10 and voltage measuring device 11 are connected to a control device 12. The control device 12 includes a microcomputer. A storage device 13 in which various data items are stored is connected to the control device 12. The display input device 5 shown in FIG. 1 has an input device 15 and a display device 16 incorporated therein in the form of electric circuits. The display input device 5 is connected to the control device 12 with an input/output control device 17, which controls the input device 15 and display device 16, between them. A power supply 14 supplies power to the control device 13 and the other devices. When the Internet is accessed through a modem 8, information available on the Internet can be displayed on the display device 16.

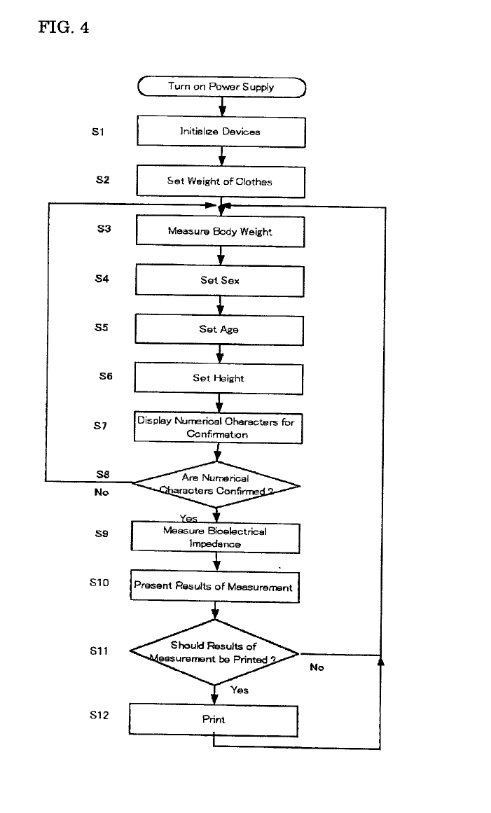

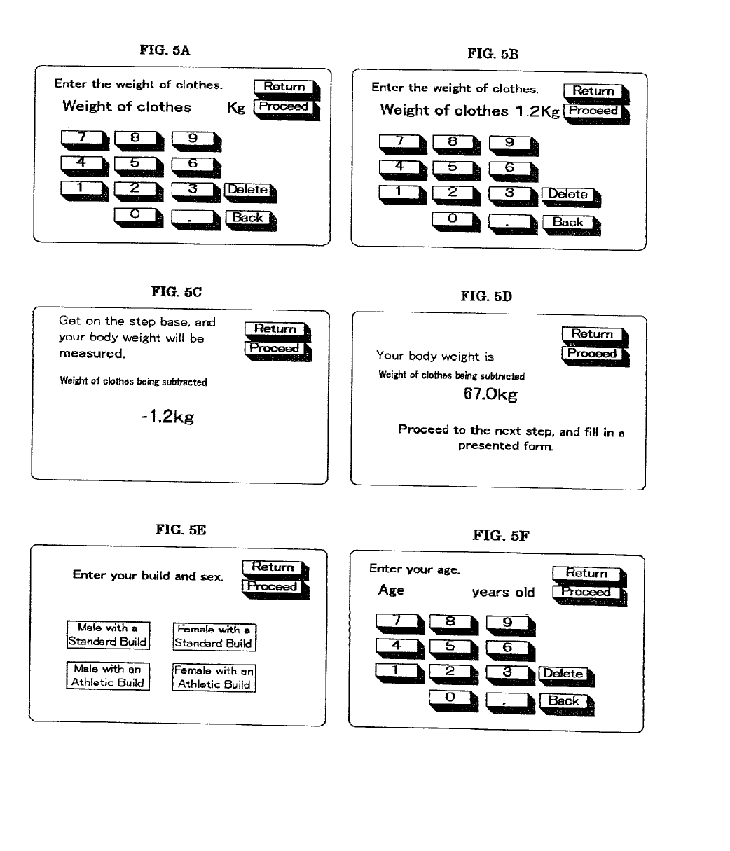

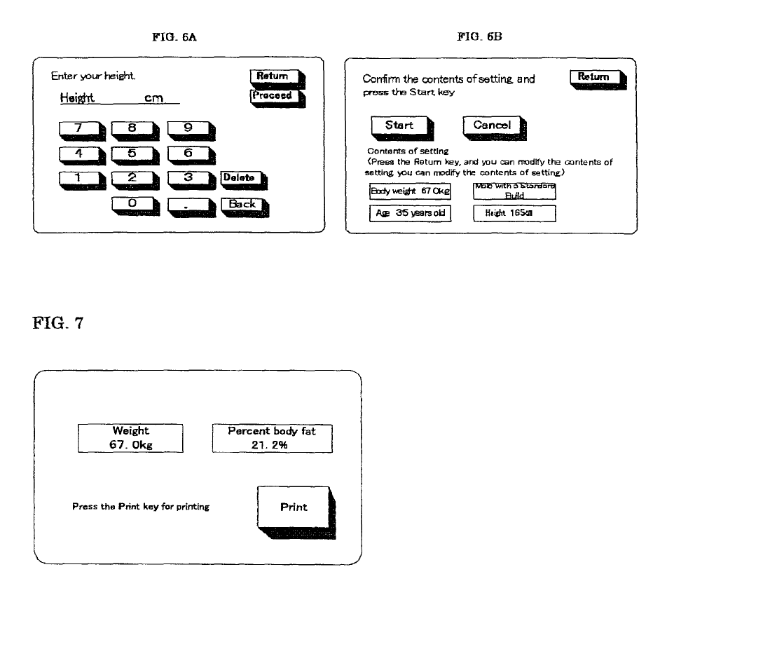

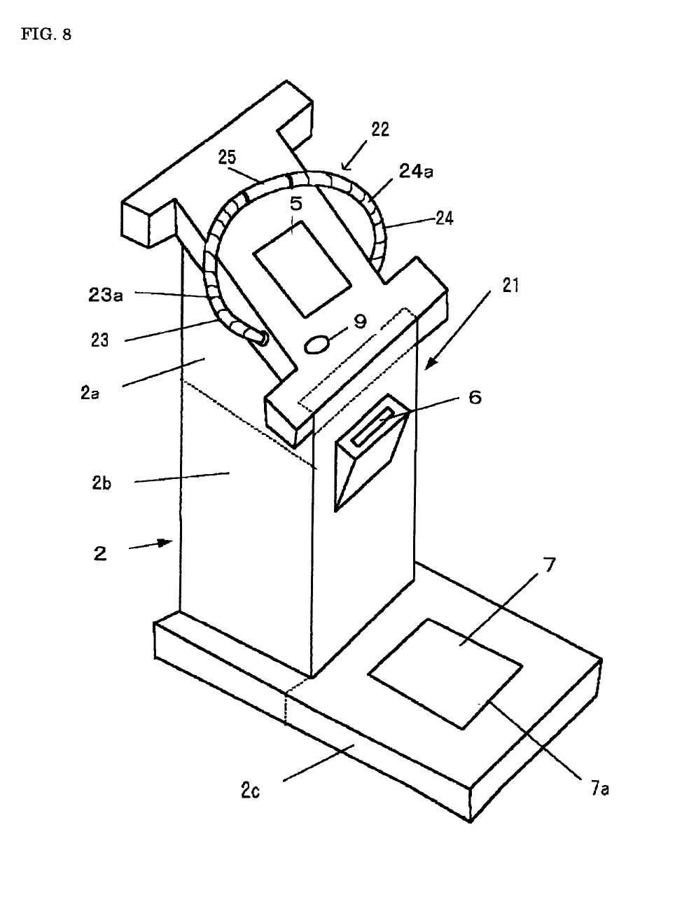

Next, actions to be performed in the measuring apparatus 1 will be described in conjunction with the flowchart of FIG. 4 as well as FIG. 5 and FIG. 6 that show screen images. First, a power switch 9 on the measuring apparatus 1 is turned on. All the electric devices are initialized at step S1. A weight-of-clothes input mode is -invoked at step S2, whereby a screen image shown in FIG. 5A appears. In this mode, a message prompting a user to enter the weight of his/her clothes and an input keyboard used to enter the weight of clothes are displayed on the display device 16. A user who is a subject uses the numerical keys of the input keyboard to enter the weight of his/her clothes. For example, a weight of 1.2 kg is entered by touching the screen of the display device with a finger. A screen image shown in FIG. 5B then appears. If a Proceed key is pressed, control is passed to step S3. If an incorrect value is entered, a Delete key may be used to delete the entered value. Otherwise, a Back key may be used to delete entered numerical characters one by one, and a correct value may be entered.

Step 3 is a step of invoking a mode in which a body weight is measured. As shown in FIG. 5C, the user is prompted to stand on the step base 7a of the weighing device 7. The weight of clothes entered at step 2 is presented with a negative sign so that the weight of clothes will be subtracted from a measured body weight. When the user stands on the step base 7a, his/her body weight is measured on the weighing device 7. Numerical characters representing the body weight are displayed as shown in FIG. 5D. A message prompting the user to proceed to step S4 is also displayed. If the Proceed key is pressed at this time, a build and a sex can be entered as shown in FIG. 5E. For example, Male with a Standard Build is pressed and the Proceed key is pressed. Control is then passed to step S5. At step S5, an age is entered. As shown in FIG. 5F, the numerical keys are used to enter the user's age, and the Proceed key is pressed. Control is then passed to step S6. At step S6, a height is entered. As shown in FIG. 6A, the user uses the numerical keys to enter his/her height, and then presses the Proceed key. Control is then passed to step S7. The user data measured or entered from step S3 to step S6 is presented on the display device 16 (FIG. 6B). At this time, if a Return key or Cancel key is pressed, control is returned to step S2. Input and measurement are restarted. If a Start key is pressed, control is passed to step S9. This triggers measurement of bioelectrical impedance relative to current flowing between the user's hands.

At step S9, the user brings the thumb of his/her left hand into contact with the electrode 3a. The palm, index finger, middle finger, and third finger of the user's left hand are brought into contact with the electrode 3b. The fingers of the user's right hand are arranged in the same manner. When the width of the electrodes 3a and 4a is determined appropriately, and the user faces the front panel of the apparatus 1, and grabs the electrode member 3 with his/her left hand and the electrode member 4 with his/her right hand, unintentionally the user's fingers come into contact with the electrodes as mentioned above. Moreover, since the electrode members are shaped like rods and arranged lengthwise, an error derived from a change in a posture caused by a difference in a height can be minimized. Thereafter, the control device 13 instructs the current supplying device 10 to supply alternating current to the electrodes 3a and 4a. The voltage measuring device 11 measures voltage at the electrodes 3b and 4b. The control device 13 calculates a bioelectrical impedance value from the supplied current value and the measured voltage values.

At step S10, the body weight and the percent body fat are presented as shown in FIG. 7. If a Print key is pressed at this time (step S11), the printer 6 produces a printout at step S12. Unless the Print key is pressed, control is returned to step S2.

The present measuring apparatus has an advising feature to introduce a diet or a medicine that is selected based on the percent body fat. Moreover, the location of the diet or medicine in the store, the price thereof, and the inventory thereof can be presented. If no good is in stock in the store, the inventories of the diet or medicine in the other franchised stores can be acquired over the Internet accessed through the modem.

Moreover, servers installed in sporting clubs or the like may be accessed over the Internet through the modem. Thus, a sporting club or an esthetic club selected based on the percent body fat can be introduced.

The measured values concerning individuals may be stored in the storage device 13 connected to the measuring apparatus or a server connected on the Internet, whereby a time-sequential change in each measured value can be presented. If the measured values are stored in the server connected on the Internet, a time-sequential change in each measured value can be presented on the measuring apparatus installed in any of franchised stores.

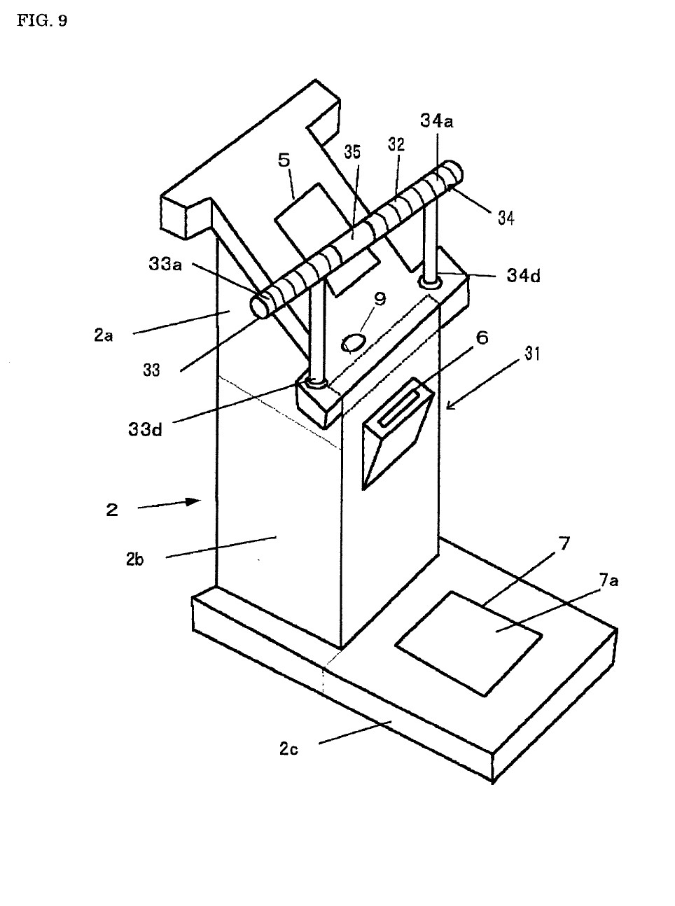

Next, a second embodiment of a bioelectrical impedance measuring apparatus in accordance with the present invention will be described below. FIG. 8 is a perspective view showing the appearance of the second embodiment of the bioelectrical impedance measuring apparatus in accordance with the present invention. In FIG. 8, components that play the same roles as those shown in FIG. 1 bear the same reference numerals. A measuring apparatus 21 has, as shown in FIG. 8, electrode members 23 and 24 in place of the electrode members 3 and 4 employed in the first embodiment. The electrode members 23 and 24 are linked with an insulator 25 between them, thus forming a substantially circular electrode member 22. The electrode member 23 has the same cross section as the electrode member 3 employed in the first embodiment. The electrode 23 consists of electrodes 23a and 23b, and an insulator 23c. Since the electrode members have the circular shape, users having different heights or having shoulders of different breadths can share the measuring apparatus. The other external components and the internal components are identical to those of the first embodiment. A measuring procedure and actions performed in the present embodiment are generally identical to those performed in the first embodiment.

Next, a third embodiment of a bioelectrical impedance measuring apparatus in accordance with the present invention will be described below. FIG. 9 is a perspective view showing the appearance of the third embodiment of the bioelectrical impedance measuring apparatus in accordance with the present invention. In FIG. 9, components that play the same roles as those shown in FIG. 1 bear the same reference numerals. A measuring apparatus 31 has electrode members 33 and 34 in place of the electrode members 3 and 4 employed in the first embodiment. The electrode members 33 and 34 are linked with an insulator 35 between them, thus forming a horizontal rod-like electrode member 32. The electrode member 33 is connected to a control panel 36 over a pipe 33d. A ratchet that is not shown is formed at the joint between the pipe 33d and control panel 36, whereby the pipe 33d can pivot with the joint as a fulcrum. The ratchet enables the pipe 33d to pivot upwards but not to pivot downwards. once the ratchet is disengaged, the pipe 33d pivots downwards. Since the electrode members are shaped like horizontal rods, users having extremely broad or narrow shoulders can share the measuring apparatus.

The other external components and the internal components are identical to those employed in the first embodiment. A measuring procedure and actions performed in the present embodiment are generally identical to those performed in the first embodiment.

According to the present invention, a weighing device is incorporated and bioelectrical impedance is measured relative to current flowing between both hands. A user is therefore not required to have his/her feet bared. Nevertheless, the bioelectrical impedance can be measured highly precisely. Moreover, since electrodes are shaped like rods and arranged lengthwise, an error derived from a change in a posture caused by a difference in a height can be minimized.

What is claimed is:

1. A bioelectrical impedance measuring apparatus comprising:
a housing;
rod-like electrode members each having a plurality of electrodes and being included in the upper part of said housing;
a display device having the capability of an operator panel and lying between said electrode members; and
a weighing device included in the lower part of said housing,
wherein said housing accommodates:
a current supplying device for supplying current to said electrodes;
a voltage measuring device for measuring voltage at said electrodes; and
an arithmetic means for calculating a bioelectrical impedance value from the supplied current value and the measured voltage values.

2. A bioelectrical impedance measuring apparatus according to claim 1, wherein said electrode members are located at both side edges of said housing.

3. A bioelectrical impedance measuring apparatus according to claim 1, wherein said electrode members have a circular shape.

4. A bioelectrical impedance measuring apparatus according to claim 1, wherein said electrode members are shaped like horizontal rods.
